(19) United States
(12) Patent Application Publication
(10) Pub. No.: US 2005/0208006 A1
(43) Pub. Date: Sep. 22, 2005

(54) Hair cosmetic composition comprising a nanoobject of elongate form made of crosslinked synthetic polymer, method employing this composition, and uses

(76) Inventors: Estelle Mathonneau, Paris (FR); Isabelle Rollat, Paris (FR); Nathalie Mougin, Paris (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 11/009,038

(22) Filed: Dec. 13, 2004

Related U.S. Application Data
(60) Provisional application No. 60/562,553, filed on Apr. 16, 2004.

(30) Foreign Application Priority Data
Dec. 11, 2003 (FR) 0314541

(51) Int. Cl. A61K 7/06

(52) U.S. Cl. 424/70.1

(57) ABSTRACT

Disclosed herein are a cosmetic composition for the cosmetic treatment of keratin fibers, which comprises, in a cosmetically acceptable medium, at least one nanoobject of elongate form made of at least one crosslinked synthetic polymer, and methods of cosmetic treatment of keratin fibers comprising applying this composition to the keratin fibers to impart body to the hairstyle.

This application claims benefit of U.S. Provisional Application No. 60/562,553, filed Apr. 16, 2004.

The present disclosure relates to a cosmetic composition for cosmetic treatment of keratin fibers, for example, human keratin fibers such as the hair, which comprises, in a cosmetically acceptable medium, at least one nanoobject of elongate form made of at least one crosslinked synthetic polymer. The present disclosure further relates to methods of cosmetic treatment of keratin fibers, which comprise applying this composition to keratin fibers, such as the hair, to impart body to the hairstyle, and to the uses of this cosmetic composition to impart body to the hairstyle.

The most widespread styling products intended for enhancing the body of the hairstyle that are on the market are based on film-forming polymers. Upon application, these products most often allow the desired effect to be obtained; however, the effect obtained may fade rapidly and disappear completely at the first shampooing. These products may also have the drawback of impairing the hair and giving it a loaded feel.

Another technique for enhancing the body of the hairstyle includes carrying out a permanent shaping (“perming”) treatment on the keratin fibers: these “perms” comprise applying a reducing agent to the keratin fibers, subjecting them to a set under mechanical tension, generally using rollers, and then applying an oxidizing agent to them. This technique may allow the body of the hairstyle to be enhanced durably, but may have at least one of the drawbacks of modifying the shape of the hairstyle and the level of curliness, and also of degrading the keratin fiber.

Surprisingly, the present inventors have discovered that the use of nanoobjects of elongate form made of at least one crosslinked synthetic polymer in a cosmetic composition can allow the body of the hairstyle to be durably augmented.

Accordingly, disclosed herein is a cosmetic composition for the cosmetic treatment of keratin fibers, for example, human keratin fibers such as the hair, comprising, in a cosmetically acceptable medium, at least one nanoobject of elongate form made of at least one crosslinked synthetic polymer.

The composition as disclosed herein can exhibit high affinity for keratin fibers. This composition can allow a substantial enhancement of the body of the hairstyle, which may remain over one or more shampooings.

Furthermore, owing to their nature, the compositions as disclosed herein do not contain agents which degrade the keratin fibers. The keratin fibers are not, consequently, impaired by repeated applications of the inventive compositions, and their feel is not modified.

These compositions may also possess good cosmetic properties in terms of softness and lightness of the hairstyle.

As used herein, the term “nanoobject of elongate form made of at least one crosslinked polymer” means any three-dimensional object comprising at least one crosslinked polymer, whose dimension along one of the axes (length) is greater than the dimensions along the two other axes (cross-section).

These nanoobjects of elongate form made of at least one crosslinked polymer are, for example, nanotubes having a circular, ellipsoidal or hexagonal cross-section, with external diameters or axes ranging, for example, from 0.1 to 200 nm, or helixes with an external diameter ranging, for example, from 0.1 to 200 nm.

The length of these nanoobjects of elongate form made of at least one crosslinked synthetic polymer ranges, for example, from 10 nm to 10 μm.

The ratio of the length to the cross-section of the nanoobject is greater than 1:1, such as greater than 2:1, further such as greater than 3:1. In one embodiment, this ratio is less than 100 000:1.

The polymer used to obtain the nanoobject is chosen from synthetic polymers.

As used herein, a “synthetic polymer” is a polymer obtained by chemical or electrochemical synthesis (free-radical addition polymerization, polycondensation, ring-opening polymerization or metathesis polymerization). Crosslinking may take place chemically or under the action of photochemical radiation, such as under the action of UV or of temperature.

This polymer may be a homopolymer or a copolymer. For example, homopolymers or copolymers deriving from the free-radical addition polymerization of monomers comprising at least one unit chosen from ethylenic, vinylic, allylic, (meth)acrylate and (meth)acrylamide units and units derived therefrom may be used.

In one embodiment, copolymers chosen from vinyl/(meth)acrylate, vinyl/(meth)acrylamide, vinyl/(meth)acrylate/(meth)acrylamide, olefin/vinyl and (meth)acrylate/(meth)acrylamide copolymers are used.

Further, for example, homopolymers or copolymers based on monomers chosen from vinyl acetate, styrene, vinylpyrrolidone, vinylcaprolactam, stearyl (meth)acrylate, lauryl(meth)acrylate, vinyl laurate, butyl(meth)acrylate, ethylhexyl (meth)acrylate, crotonic acid, (meth)acrylic acid, sodium styrenesulphonate, dimethyldiallylamine, vinylpyridine, dimethylaminoethyl(meth)acrylate, dimethylaminopropyl(meth)acrylamide and salts thereof may be used.

The polymers as disclosed herein may also be chosen, for example, from:
polycondensates of polyurethane and/or polyureas, of aliphatic polyesters, aliphatic polyamides and of copolymers thereof, such as polyurethane/urea and polyester/amide,
polymers obtained by ring opening, such as polyethylene oxide and polypropylene oxide polyethers, polylactides and their polyethylene oxide/polypropylene oxide copolymers; polyesters such as polycaprolactone; and polyoxazolines such as poly(2-methyloxazoline) and poly(2-ethyloxazoline),
siloxane copolymers, for example, polydimethylsiloxane (PDMS), polymethylphenylsiloxane,
polymers obtained by metathesis, such as poly(norbornene) and its copolymers, and
polymers obtained by cationic polymerization, such as polyvinyl alkyl ethers, for instance polyvinyl methyl ethers, and
copolymers of these various types of polymers, such as polysiloxane/polyethylene oxides.

These polymers may, where appropriate, be functionalized so as to endow them with the characteristic of solubility or dispersability in water and/or ethanol, or in carbon oils, ester oils, fluoro oils or silicone oils.

Examples of the homopolymer, which may be used herein, include polypyrrole, polyaniline, polyethylenedioxythiophene, polymethyl methacrylate, polytetrafluoroethylene, poly-L-lactide/palladium acetate, poly(p-xylene), polymerized [2.2]paracyclophane, polymerized 1,4-quinodimethane, polystyrene, polypropylene, poly-(p-phenylenebenzobisoxazole) and polyaryleneethynylene.

Examples of the copolymer, which may be used herein, include polystyrene-b-poly(2-cinnamoylethyl methacrylate)s (PS-b-PCEMA), polyacid-acrylate-b-poly(2-cinnamoylethyl methacrylate)s (PAA-b-PCEMA), polybutylacrylate-b-poly(2-cinnamoylethyl methacrylate)s (PBtA-b-PCEMA) and polyimine-b-poly(2-cinnamoylethyl methacrylate)s (PI-b-PCEMA).

The following list provides examples of synthetic polymer nanoobjects known in the art and suitable for use in the compositions as disclosed herein. This list should not on any account be interpreted as limiting the invention.

In the following list, the polymer nanoobjects are grouped in accordance with the method of synthesis used for obtaining them.

The five techniques presented below may be used for preparing homopolymer or copolymer nanoobjects.

a) By Polymerization in the Pores of a Membrane.

This mode of synthesis allows hollow or solid nanoobjects to be obtained. According to this technique, the shape is fixed by the crosslinking.

This technique has made it possible to obtain nanotubes of polypyrrole (J. Duchet, R. Legras, S. Demoustier-Champagne, Synth. Met., 98 (1998) 113; S. Demoustier-Champagne, J. Duchet, R. Legras, Synth. Met., 101 (1999) 20; V. P. Menon, J. Lei, C. R. Martin, Chem. Mater., 8 (1996) 2382), nanotubes of polyaniline (Z. Cai, J. Lei, W. Liang, V. Menon, C. R. Martin, Chemical Materials, 3, (1991), 960; J. Duchet, R. Legras, S. Demoustier-Champagne, Synthetic Metals, 98 (1998) 113), and nanotubes of polyethylene-dioxythiophene (J. L. Duval, P. Rétho, G. Louarn, C. Godon, C. Marhic, S. Demoustier-Champagne, S. Garreau, Matériaux, (2002)).

The nanoobject synthesis methods described in the bibliographic references cited in the present application are incorporated by reference.

These syntheses take place by an oxidative polymerization either by an electrochemical route or using an oxidizing agent (chemical route) on different types of membranes (“Template synthesis on track-etched membranes”). This mode of synthesis is described in the following documents: S. Demoustier-Champagne, E. Ferain, R. Legners, C. Jérôme, R. Jérôme, Eur. Polym. J., 34 (1998) 1767; S. Demoustier-Champagne, P-Y Staveux, Chem. Mater., 11 (1999) 829; and C. R. Martin, Science, 266 (1994) 1961-1966.

The most widely used synthesis method is the electrochemical-route method in the pores of a gold-covered polycarbonate membrane (anode). By controlling the polymerization time, tubes with thin or thick walls can be obtained.

The membrane is immersed in a solution comprising the monomer and LiClO₄ and the gold-covered membrane and the reference electrode are subjected to a voltage in order to carry out the polymerization. All monomers which can be polymerized oxidatively and have at least one solubilizing functional group can be used. As used herein, the term “solubilizing functional group” means that the monomer includes a functional group which renders the monomer soluble in the polymerization medium.

The size (length, diameter, thickness) of the nanoobjects depends on the pore size of the membrane used, on the monomer used in forming the nanoobject, and on the polymerization time. R. V. Parthasarathy, C. R. Martin, Chem. Mater., 6 (1994) 1627 shows that with polypyrrole a thick tube is formed rapidly; while with polyaniline the polymerization is slower and leads to the formation of thin tubes.

According to this technique, when the nanoobject has been formed, the membrane is removed by dissolution in a solvent such as dichloromethane as a mixture with a surfactant such as dodecyl sulphate. The mixture is subsequently subjected to ultrasound for an hour in order to obtain complete removal of the polycarbonate.

The type of membrane used is not critical, but it should be able to dissolve in an appropriate solvent.

One variant includes the technique of “melt wetting of macroporous templates”, which is described in the following documents: M. Steinhart, J. Wendorff, from Philips University, Marburg; R. Wehrspohn, U. M. Gösele, from the Max-Planck Institute; Wehrspohn and U. Gösele, from the Max-Planck Institute of Microstructure Physics, Science (296), 1997 (2002); and S. Demoustier-Champagne, J. Duchet, R. Legras, Synthetic Metals, 101 (1-3), (1999) 20-21.

This variant makes it possible to obtain nanoobjects made of at least one polymer chosen from polytetrafluoroethylene (PTFE), polymethyl methacrylate (PMMA) and poly-L-lactide/palladium acetate. The membrane used has a very small pore size, and this membrane is generally made of alumina, oxidized silicone or glass. The pores of this membrane are impregnated with a liquid comprising the polymer, and when the nanoobject has been formed, the membrane is dissolved in a KOH solution.

b) By Polymerization Around a Support.

This technique allows generally hollow nanoobjects to be obtained, and according to this technique, the shape is fixed by the crosslinking.

According to this mode of synthesis, the monomer used for the polymerization has at least one functional group which permits polymerization and, in some embodiments, a functional group which promotes the interaction of the monomer with the support via Van der Waals bonds, hydrogen bonds, ionic bonds, covalent bonds, π-π bonds, etc.

The size (length, diameter, thickness) of the nanoobjects depends on the support (cross-section, length), on the monomer used to form the nanoobject, on the amount of the monomer and on the polymerization time.

The supports are of elongate form and are generally chosen from:
natural nanofibrils of any type: cellulose, protein, silk, etc.;
synthetic nanofibers (for example, polyamide);
carbon nanotubes, which are or are not functionalized (J. Chen, Science, 282 (1998) 95-98) (by, for example, carboxyl groups, hydrophobic functional groups, fatty amides of formula CONH-4-C₆H₄(CH₂)₁₃CH₃); and
nanotubes of type BₓC_{y}N_{z}. (Z. Weng-Sieh, Physical Review B, 16 (1995) 11229-32; E. Hernandez, C. Goze, P. Bernier, A. Rubio, Physical Review Letter, 80(20) (1998) 4502-05).

The uses of fibers as a synthesis support or “fiber template support” have been presented in the following documents: H. Hou, Z. Jun, A. Reuning, A. Schaper, J. Wendorff, A. Greiner, Macromolecule, 35(7), (2002) 2429-31; and H. Dong, W. E. Jr. Jones, Polymeric Materials Science and Engineering, 87 (2002) 273-4.

Polyamides 4/6 several tens of nanometers in diameter (obtained by electrospinning a solution of PA/HCOOH (8%) in the presence of pyridine) and fibers of poly(L-lactide) with a slightly greater diameter (obtained by electrospinning a solution of PLA (1.5%)/H₂Cl₂ in the presence of Pd(OAc)₂) served as a support for obtaining nanotubes from polymers such as poly(p-xylene), [2.2]paracyclophane (dimer) and 1,4-quinodimethane (monomer). The supports are removed by solvent extraction.

The documents S. Kumar, T. D. Dang, F. E. Arnold, A. R. Bhattacharyya, B. G. Min, X. Zhang, R. A. Vaia, C. Park, W. W. Adams, R. H. Hauge, R. E. Smalley, S. Ramesh, P. A. Willis, Macromolecules, 35 (2002) 9039-43; R. Andrews, D. Jacques, A. M. Rao, T. Rantell, F. Derbyshire, Y. Chen, J. Chen, R. C. Haddon, Appl. Phys. Lett., 75 (1999) 1329; D. Quin, E. C. Dickey, R. Andrews, T. Rantell, Appl. Phys. Lett., 76 (2000) 20; R. Haggenmueller, H. H. Gommans, A. G. Rinzler, J. E. Fischer, K. I. Winey, Chem. Phys. Lett., 330 (2000) 219; and S. Kumar, H. Doshi, M. Srinivasrao, J. O. Park, D. Aschiraldi, Polymer, 43 (2002) 1701 describe syntheses of nanoobjects by polymerization around a carbon nanotube.

c) By Polymerization Around a Shape Obtained Using a Surfactant.

This technique allows generally hollow tubes to be obtained, and according to this technique, the shape is fixed by the crosslinking.

The size (length, diameter, thickness) of the nanotubes depends on the surfactant, on the monomer used to form the nanotube, on the amount of the monomer and on the polymerization time.

This technique allows polypyrrole nanotubes to be obtained (J. Jang, H. Yoon, Chem. Comm., (2003) 720-21) and employs an inverse emulsion in an a polar solvent. The surfactant used therein is sodium bis(2-ethylhexyl)sulphosuccinate in hexane, and this surfactant forms an inverted micellar structure with hydrophilic groups within the micellar structure. The polypyrrole polymerizes on the outside of the tube. Solvent extraction of the surfactant allows polypyrrole tubes to be obtained.

d) By Passing a Polymer Solution into the Pores of a Membrane.

This technique allows generally solid tubes to be obtained, and according to this technique, the shape is not fixed, since it can be perturbed by adding a solvent or an additive.

The size (length, diameter, thickness) of the nanotubes depends on the size of the pores of the membrane and on the polymer used to form the nanotube.

The monomers used for the polymerization should have functional moieties which promote self-assembly (by π-π bonds, hydrogen bonds, by acid-base interaction, by dipole-dipole interaction, etc.).

This technique is disclosed, for example, in the document J. N. Wilson, C. G. Bangcuyo, B. Erdogan, M. L. Myrick, U. H. F. Bunz, Macromolecules, 36 (2003) 1426-28. Polyarylene-ethynylene nanotubes are obtained by passing polymer flows through porous membranes. The self-assembly properties of the polyaryleneethynylenes are utilized to form nanotubes in the pores of the membranes. These nanotubes do not exhibit a fixed shape.

e) Synthesis of Nanoobjects from Block Copolymers

Block copolymer nanotubes can be obtained by physical combinations of the block copolymer in a solvent or a mixture of solvents, the solvent possessing preferential affinity for one of the blocks of the copolymer. This phase separation is governed, for example, by the presence of a solubilizing group on this block. Relative to the other synthesis methods presented herein, the formation of the nanotubes passes via crosslinking of one of the blocks, which allows the shape of the nanotube to be fixed and a stable shape and structure to be conferred, irrespective of the medium in which the polymer nanotubes are integrated.

The polymer used for producing nanotubes by this synthesis route can be, for example, a diblock AB or a triblock ABA, BAB or ABC. At least one of the blocks, A, B or C, should be able to be crosslinked. The crosslinked block may be that forming the core of the nanotube or the sheath of the nanotube. Monomers used to form the crosslinked block are chosen from those which have crosslinking functional groups of type X or Y, wherein the bonds formed are of X—X, Y—Y or X—Y type. The functional groups X are chosen, for example, from: XHₙ wherein X═O, N, S or COO and n=1 or 2, for example, alcohols, amines, thiols and carboxylic acids. The functional groups Y are chosen, for example, from:
epoxide;
aziridine
activated and non-activated double bonds, such as ethylenes and derivatives thereof, for example, butadiene, isoprene, acrylic and methacrylic esters, vinyl esters, vinyl ethers, cinnamic esters, styrenes and derivatives thereof, crotonic and maleic esters, anhydrides and chlorides of unsaturated acids, crotonic acid, and cinnamic acid;
aldehydes;
acetals, hemiacetals;
aminals, hemiaminals;
ketones, alpha-hydroxy ketones, alpha-halo ketones;
lactones, thiolactones;
isocyanate;
thiocyanate;
imines;
imides, such as succinimide and glutimide;
N-hydroxysuccinimide esters;
imidates;
thiosulphate;
oxazine and oxazoline;
oxazinenium and oxazolinium;
C₁ to C₃₀ alkyl and C₆ to C₃₀ aryl and aralkyl halides of formula RX, wherein X═I, Br or Cl;
halides of a ring chosen from unsaturated rings, carbon rings and heterocyclic rings, such as chlorothiazines, chloropyrimidines, chloroquinoxalines and chlorobenzotriazoles; and
sulphonyl halides: such as RSO₂Cl and RSO₂F, wherein R is chosen from C₁ to C₃₀ alkyl radicals.

The crosslinking may be performed using an intermediate compound comprising more than two X or Y functional groups.

The size (length, diameter, thickness) of the nanotubes depends on the choice of blocks, on the size of the blocks, on the proportion of the blocks and on the choice of solvents.

The solubilizing moieties are, for example, chosen from:
carboxyl (—COOH) and carboxylate radicals (—COO³¹ M⁺, wherein M is chosen from an alkali metal such as sodium and potassium, an alkaline earth metal, an organic amine such as a primary, secondary, or tertiary amine, and an alkanolamine, and an amino acid),
sulphonic (—SO₃H) and sulphonate radicals (—SO₃⁻M⁺, wherein M has the same definition as above),
primary, secondary and tertiary amine radicals,
a quaternary ammonium radical such as—NR′₃⁺Z⁻ wherein Z=Br, Cl, or alkyl(C₁-C₄)—OSO₃ and R's, which are identical or different, are each chosen from linear and branched C₁ to C₂₀ alkyl radicals, and radicals two of which form a heterocycle with the nitrogen,
a hydroxyl radical, and
poly-C₂-C₃ alkene oxide radicals.

The carboxylic or sulphonic acid functional groups may or may not be neutralized with a base, such as sodium hydroxide, 2-amino-2-methylpropanol, triethylamine and tributylamine.

The amine radicals may or may not be neutralized with an inorganic acid, such as hydrochloric acid, or with an organic acid, such as acetic acid or lactic acid.

Moreover, it should be noted that the solubilizing radicals may be connected to the ring via a spacer group such as a radical —R″, —OR″, —OCOR″— or —COOR″— wherein R″ is chosen from linear and branched C₁-C₂₀ alkyl radicals optionally comprising at least one heteroatom, such as oxygen.

In one embodiment, the polymer used to form the nanoobject comprises at least one solubilizing group per repeating unit (monomer).

Examples of such nanotubes are described in G. Liu, Handbook of Nanostructured Materials and Technology, (2000), pp. 475-500: poly(2-cinnamoylethyl methacrylate) (PCEMA): PS-b-PCEMA (polystyrene-b-poly(2-cinnamoylethyl methacrylate)), PAA-b-PCEMA (polyacid-acrylate-b-poly(2-cinnamoylethyl methacrylate)), PBtA-b-PCEMA (polybutyl acrylate-b-poly(2-cinnamoylethyl methacrylate)), PI-b-PCEMA (polyimine-b-poly(2-cinnamoylethyl methacrylate)). Other examples include block copolymers in which one block is chosen from polydimethylaminoethyl methacrylate, PAA, a polymethacrylic acid, PEO, and silicone, such as polydimethylaminoethyl methacrylate-b-cinnamoyl, PAA-b-poly(cinnamoyl-co-butyl acrylate or co-styrene or co-methyl methacrylate), silicone-b-cinnamoyl, PS-b-(polyCEMAcoHEMA).

The size and shape of the tubes depend on the choice of the monomers in the blocks and on the proportion of the blocks in the block polymer. Hollow or solid tubes may be obtained, depending on the choice of polymers and solvents.

According to the technique used, the nanoobject may be solid or hollow.

In the case of a hollow nanoobject, it may be closed at its ends or open; moreover, it may comprise at least one filling compound, which may be chosen, for example, from gases, liquids and solids, wherein the at least one filling compound is different from the polymer forming the nanoobject.

On the inside or on the outside of their wall, the nanoobjects may absorb or adsorb at least one molecule chosen, for example, from linear and branched polymers, doped and undoped fullerenes, carbon nanotubes and cosmetic additives whose size is compatible with the nanoobjects.

The outside surface of the nanoobjects may also be functionalized in order to enhance the affinity of the nanoobjects with the keratin fiber or with the medium.

A “functionalized surface” as used herein is an outside surface which is modified by the presence of functional groups allowing physical or chemical interaction with one another, with the fiber or with the medium in which the nanoobjects are placed.

Enhancing the affinity of the nanotubes for the keratin fiber is accomplished, for example, by groups which possess a certain reactivity with the amino acids constituting the keratin material. For example, the functional group or groups able to create one or more covalent chemical bonds with the keratin fiber are chosen from groups capable of reacting with thiols, disulphides, carboxylic acids, alcohols and amines.

The cosmetic composition according to the present disclosure comprises, for example, from 0.00001% to 30% by weight such as from 0.001% to 10% by weight of nanoobjects of elongate shape made of at least one crosslinked polymer, relative to the total weight of the composition.

The cosmetic composition according to the present disclosure may comprise at least one nanoobject of elongate form; in other words, it may comprise nanoobjects all composed of the same polymer or composed of different polymers.

The cosmetically acceptable medium generally comprises water, at least one organic solvent or a mixture of water and at least one organic solvent.

The at least one organic solvent may be chosen from the following solvents: C₁-C₄ alcohols such as ethanol and isopropanol, C₅-C₁₀ alkanes, ketones such as acetone and methyl ethyl ketone, ethers such as dimethoxyethane and diethoxyethane, esters such as methyl acetate, ethyl acetate and butyl acetate, fatty alcohols, modified and non-modified polyols such as glycerol, volatile and non-volatile silicones, mineral, organic and vegetable oils, waxes, fatty acids and mixtures thereof.

The cosmetically acceptable medium may be provided in an emulsified form, and the nanoobjects may also be encapsulated.

The cosmetic composition according to the present disclosure may further comprise at least one cosmetic additive.

A “cosmetic additive” as used herein is chosen from oxidizing agents, reducing agents, fixative polymers in soluble, dispersed and microdispersed forms, thickening polymers, nonionic, anionic, cationic and amphoteric surfactants, conditioning agents, softeners, moisturizers, emollients, antifoams, ceramides and pseudoceramides, vitamins and provitamins, including panthenol, non-silicone fats such as vegetable, animal, mineral and synthetic oils, volatile and non-volatile, linear and branched silicones, with or without organic modification, water-soluble and fat-soluble, silicone and non-silicone sunscreens, organic and inorganic pigments, colored and non-colored, permanent and temporary dyes, mineral fillers, clays, minerals, colloids, nacres and opacifiers, proteins, sequestrants, plasticizers, solubilizers, acidifying agents, alkalifying agents, hydroxy acids, penetrants, perfumes, perfume solubilizers (peptizers), preservatives and anti-corrosion agents.

The at least one cosmetic additive is present in the cosmetic composition as disclosed herein in an amount ranging from 0% to 20% by weight relative to the total weight of the cosmetic composition.

The person skilled in the art will take care to select the optional additives and their amount such that they are not detrimental to the properties of the compositions of the present disclosure.

The composition according to the present disclosure may be provided in the form of a lotion, spray, foam, shampoo or conditioner.

The composition as disclosed herein may also be present in the form of a lacquer, in which case it is applied using a propellant gas. This propellant gas comprises the compressed or liquefied gases which are commonly used for the preparation of aerosol compositions.

The present disclosure further provides a method of cosmetic treatment for the purpose of imparting body to the hairstyle, comprising applying the composition as disclosed herein to keratin fibers, for example, human keratin fibers such as the hair.

The present disclosure also provides for the use of the cosmetic composition as disclosed herein on keratin fibers, for example, human keratin fibers such as the hair, for the purpose of imparting body to the hairstyle.

The composition may be applied to dry hair (non-rinse product) or to wet hair (rinse-off product). When the composition is applied to wet hair, the time of application to the hair before rinsing ranges from a few seconds to a few minutes, generally from 5 seconds to 20 minutes.

The keratin fibers may also be heated before, during or after the application of the composition as disclosed herein.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The examples which follow illustrate the invention without limiting its scope.

### EXAMPLE 1

Synthesis of poly-tert-butyl Acrylate (PATB)-b-PCEMA Nanotubes:

tert-Butyl acrylate was polymerized at −78° C. in tetrahydrofuran (THF) with sec-butyllithium as initiator. Subsequently, 1,1-di-phenylethylene (DPE) and lithium chloride (3 mole equivalents of sec-butyllithium) were added. The DPE reacted with the poly-tert-butyl acrylate anion to form a PATB-DPE anion. The other block was prepared by polymerizing trimethylsiloxyethyl methacrylate (HEMA-TMS) with the PATB-DPE anion. The trimethylsilyl groups were removed by hydrolysis in the presence of THF/methanol. This gave a PATB-block-poly(2-hydroxyethyl methacrylate (PATB-b-PHEMA) diblock. Reacting cinnamoyl chloride in the presence of pyridine gave the diblock PATB-b-PCEMA, comprising hydrolysis in a water/trifluoroacetic acid mixture. The PATB-b-PCEMA diblock has a PCEMA fraction of 24% by weight of the polymer. The solution was placed in a wide-bottomed beaker. After four days, a film was obtained, which was subsequently dried at 65° C. for three days and at 105° C. for three days. The film was then irradiated under a mercury lamp (500 W) (310 nm filter). The film thus obtained was then dissolved in water. The resulting nanotubes have a diameter of 50 nm and a length of 20 μm.

One gram of composition 1 according to the present disclosure comprising PATB-b-PCEMA nanotubes (24% by weight of PCEMA) was applied to 2.5 g of European hair.

Composition 1

| Component | Weight % |
|---|---|
| Cyclopentasiloxane | 10.0% |
| Ethoxylated (7 EO) hydrogenated castor oil | 10.0% |
| Dimethicone copolyol | 0.5% |
| Propylene glycol | 2.5% |
| Behentrimonium chloride | 1.2% |
| PATB-b-PCEMA nanotubes | 0.01% |
| Water | 75.79% |

After a leave-on time of several minutes, the hair was rinsed with water and then shaped using a hairdryer.

Composition 1 allowed a distinct increase in the body of the hair, which lasted for several days.

### EXAMPLE 2

Synthesis of Polythiophene Nanotubes:

9 g of sodium bis(ethylhexyl)sulphosuccinate (AOT) were dissolved in 40 ml of hexane at ambient temperature. The AOT formed an inverse micelle. 1 ml of a 9M aqueous solution of FeCl₃ was added to the AOT/hexane mixture. The FeCl₃ allowed micelles to be formed in the shape of rods (it allowed the secondary critical micelle concentration (CMCII) to be reduced and the ionic strength of the solvent to be increased). The polar anionic group of AOT extracted the metal cation from the aqueous phase. 0.5 g of thiophene monomer was added to the solvent. The thiophene monomer polymerized on the outside face of the micelles over three hours. By adding solvent (ethanol) and leaving the solution to precipitate for two hours, the surfactant was removed.

One gram of composition 2 according to the present disclosure comprising polythiophene nanotubes was applied to 2.5 g of European hair.

Composition 2

| Component | Weight % |
|---|---|
| Cyclopentasiloxane | 10.0% |
| Ethoxylated (7 EO) hydrogenated castor oil | 10.0% |
| Dimethicone copolyol | 0.5% |
| Propylene glycol | 2.5% |
| Behentrimonium chloride | 1.2% |
| Polythiophene nanotubes | 0.01% |
| Water | 75.79% |

After a leave-on time of several minutes, the hair was rinsed with water and then shaped using a hairdryer.

Composition 2 permitted a distinct increase in the body of the hair, which lasted for several days.

1. A cosmetic composition for cosmetic treatment of keratin fibers, comprising, in a cosmetically acceptable medium, at least one nanoobject of elongate form made of at least one crosslinked synthetic polymer.

2. The composition according to claim 1, wherein the keratin fibers are hair.

3. The composition according to claim 1, wherein the at least one nanoobject of elongate form is a nanotube.

4. The composition according to claim 3, wherein the nanotube has a circular or ellipsoidal cross-section with external diameters or axes ranging from 0.1 to 200 nm.

5. The composition according to claim 3, wherein the nanotube has a hexagonal cross-section with an external diameter ranging from 0.1 to 200 nm.

6. The composition according to claim 1, wherein the at least one nanoobject is a helix with an external diameter ranging from 0.1 to 200 nm.

7. The composition according to claim 1, wherein the at least one nanoobject has a length ranging from 10 nm to 10 μm.

8. The composition according to claim 1, wherein the ratio of the length to the cross-section of the at least one nanoobject is greater than 1:1.

9. The composition according to claim 8, wherein the ratio of the length to the cross-section of the at least one nanoobject is greater than 2:1.

10. The composition according to claim 9, wherein the ratio of the length to the cross-section of the at least one nanoobject is greater than 3:1.

11. The composition according to claim 8, wherein the ratio of the length to the cross-section of the at least one nanoobject is less than 100 000:1

12. The composition according to claim 1, wherein the at least one crosslinked synthetic polymer is obtained by chemical or electrochemical synthesis.

13. The composition according to claim 12, wherein the at least one crosslinked synthetic polymer is obtained by at least one of free-radical addition polymerization, polycondensation, ring-opening polymerization and metathesis polymerization.

14. The composition according to claim 1, wherein the at least one crosslinked synthetic polymer is a homopolymer.

15. The composition according to claim 14, wherein the homopolymer is chosen from polypyrrole, polyaniline, polyethylenedioxythiophene, polymethyl methacrylate, polytetraflurorethylene, poly-L-lactide/palladium acetate, poly(p-xylene), polymerized [2.2]paracyclophane, polymerized 1,4-quinodimethane, polystyrene, polypropylene, polyarylethylene and poly(p-phenylenebenzobisoxazole).

16. The composition according to claim 1, wherein the at least one crosslinked synthetic polymer is a copolymer.

17. The composition according to claim 16, wherein the copolymer is a block copolymer.

18. The composition according to claim 16, wherein the copolymer is chosen from polystyrene-b-poly(2-cinnamoylethyl methacrylate)s, polyacid-acrylate-b-poly(2-cinnamoylethyl methacrylate)s, polybutylacrylate-b-poly(2-cinnamoyl-ethyl methacrylate)s and polyimine-b-poly(2-cinnamoylethyl methacrylate)s.

19. The composition according to claim 1, wherein the at least one nanoobject is hollow.

20. The composition according to claim 19, wherein the at least one nanoobject comprises at least one filling compound chosen from gases, liquids and solids.

21. The composition according to claim 1, wherein at least one molecule is absorbed and/or adsorbed on the inside and/or the outside of the wall of the at least one nanoobject.

22. The composition according to claim 1, wherein the outside surface of the at least one nanoobject is functionalized.

23. The composition according to claim 1, wherein the at least one nanoobject is solid.

24. The composition according to claim 1, wherein the at least one nanoobject of elongate form is present in an amount ranging from 0.00001% to 30% by weight, relative to the total weight of the composition.

25. The composition according to claim 24, wherein the at least one nanoobject of elongate form is present in an amount ranging from 0.0001% to 10% by weight, relative to the total weight of the composition.

26. The composition according to claim 1, wherein the cosmetically acceptable medium comprises at least one solvent chosen from C₁-C₄ alcohols, C₅-C₁₀ alkanes, ketones, ethers, esters, fatty alcohols, modified and non-modified polyols, volatile and non-volatile silicones, mineral, organic and vegetable oils, waxes, and fatty acids.

27. The composition according to claim 1, further comprising at least one cosmetic additive chosen from oxidizing agents, reducing agents, fixative polymers in soluble, dispersed and microdispersed forms, thickening polymers, nonionic, anionic, cationic and amphoteric surfactants, conditioning agents, softeners, moisturizers, emollients, antifoams, ceramides and pseudoceramides, vitamins and provitamins, non-silicone fats, volatile and non-volatile, linear and branched silicones, with or without organic modification, water-soluble and fat-soluble, silicone and non-silicone sunscreens, organic and inorganic pigments, colored and non-colored, permanent and temporary dyes, mineral fillers, clays, minerals, colloids, nacres and opacifiers, proteins, sequestrants, plasticizers, solubilizers, acidifying agents, alkalifying agents, hydroxy acids, penetrants, perfumes, perfume solubilizers (peptizers), preservatives and anti-corrosion agents.

28. A method of cosmetic treatment for imparting body to a hairstyle, comprising applying to keratin fibers a composition comprising, in a cosmetically acceptable medium, at least one nanoobject of elongate form made of at least one crosslinked synthetic polymer.

29. The method according to claim 28, wherein the keratin fibers are hair.
